(19) United States
(12) Patent Application Publication
(10) Pub. No.: US 2022/0001038 A1
(43) Pub. Date: Jan. 6, 2022

(54) CONJUGATES FOR DETECTION OF AXILLARY LYMPH NODE METASTASES

(71) Applicants: H. LEE MOFFITT CANCER CENTER AND RESEARCH INSTITUTE, INC., Tampa, FL (US); UNIVERSITY OF ARIZONA, Tucson, AZ (US)

(72) Inventors: David L. Morse, Tampa, FL (US); Eugene A. Mash, Tucson, AZ (US); M. Catherine Lee, Tampa, FL (US)

(21) Appl. No.: 16/625,354

(22) PCT Filed: Jun. 21, 2018
(86) PCT No.: PCT/US18/38739

§ 371 Date: Dec. 20, 2019

Related U.S. Application Data
(60) Provisional application No. 62/522,934, filed on Jun. 21, 2017.

(51) Int. Cl. A61K 51/04; A61K 49/00; A61P 35/04

(52) U.S. Cl. CPC A61K 51/0497; A61P 35/04; A61K 49/0052; A61K 51/0482

(57) ABSTRACT

Disclosed are anti-cancer compounds and imaging agents. More specifically, the disclosed agents target carbonic anhydrase IX and XII and their use in the treatment of cancer, in particular breast cancer. Specific compounds are those that have Formula II wherein R₁ and R₂ are each independently chosen from (CH₂)ₙC≡CH and, L is a linking moiety; n is 1-8; m is 0-8; each X is independently chosen from R₃, OR₃, NH₂, NHR₃, and; and each R₃ is independently chosen from a detectable moiety or targeting moiety, with the proviso that R₁ and R₂ are not both (CH₂)₄C≡CH.

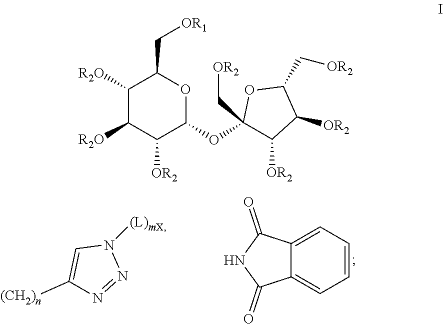

### FIELD

The subject matter disclosed herein relates generally to cancer therapy and to anti-cancer compounds and imaging agents.

### BACKGROUND

Axillary Lymph Node (ALN) status is a key determinant of prognosis and adjuvant therapy in breast cancer (Society AC. Breast Cancer Facts and Figures 2015-2016. In: American Cancer Society I, editor. Atlanta 2015). In the case of clinically node positive patients, classically a complete ALN dissection is performed. Although a longstanding standard operation, complete node dissection is morbid and confers no survival benefit (Fisher B, et al., “Ten-year results of a randomized clinical trial comparing radical mastectomy and total mastectomy with or without radiation.” New England J. Med. 1985; 312(11):674-81; Fisher B, et al., “Twenty-five-year follow-up of a randomized trial comparing radical mastectomy, total mastectomy, and total mastectomy followed by irradiation.” New England J. Med. 2002; 347(8):567-75); its primary utilities are staging and local control of disease. In clinically node negative patients, pre-operative lymph node mapping for sentinel lymph node (SLN) identification and surgical biopsy for standard histologic analysis remains the current gold standard for the evaluation of ALNs (Lyman G H, et al., “American society of clinical oncology guideline recommendations for sentinel lymph node biopsy in early-stage breast cancer.” J. Clin. Oncol. 2005; 23(30):7703-20). Numerous un-targeted agents have been used to identify SLNs: sulfur colloids, blue dyes and nanomaterials, all of which collect non-selectively into the draining lymph nodes with only transient and non-specific visualization of the lymphatic system (Mieog J, et al., “Toward optimization of imaging system and lymphatic tracer for near-infrared fluorescent sentinel lymph node mapping in breast cancer.” Annals of Surgical Oncology. 2011; 18(9):2483-91; Polom K, et al., “Breast cancer sentinel lymph node mapping using near infrared guided indocyanine green and indocyanine green-human serum albumin in comparison with gamma emitting radioactive colloid tracer.” Eur. J. Surgical Oncol. (EJSO). 2012; 38(2):137-42; Pruthi S, et al., “Pharmacokinetics of methylene blue dye for lymphatic mapping in breast cancer-implications for use in pregnancy.” Am. J. Surg. 2011; 201(1):70-5; Cai X, et al., “Encapsulated conjugated oligomer nanoparticles for real-time photoacoustic sentinel lymph node imaging and targeted photothermal therapy.” Small (Weinheim an der Bergstrasse, Germany). 2016; 12(35):4873-80; Hirche C, et al., “High rate of solitary sentinel node metastases identification by fluorescence-guided lymphatic imaging in breast cancer.” J. Surgical Oncol. 2012; 105(2):162-6). Any draining node identified intraoperatively is excised. However, surgical biopsy of SLNs is invasive and expensive, and the majority of breast cancer patients (74%) who undergo SLNB are pathologically negative (Mansel R E, et al., “Randomized multicenter trial of sentinel node biopsy versus standard axillary treatment in operable breast cancer: the ALMANAC Trial.” J. Natl. Cancer Inst. 2006; 98(9):599-609; Lyman G H, et al., “Sentinel lymph node biopsy for patients with early-stage breast cancer: american society of clinical oncology clinical practice guideline update.” J. Clin. Oncol. 2014. doi: 10.1200/jco.2013.54.1177). What are thus needed are breast cancer metastasis-targeted agents for selective nodal excision. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to cancer therapy and to imaging agents. More specifically, the subject matter disclosed herein relates to agents that target carbonic anhydrase IX and XII (CAIX and CAXII) and their use in the diagnosis and treatment of cancer. Methods of screening for new agents that target CAIX and CAXII are also disclosed.

Additional advantages will be set forth in part in the description that follows or may be learned by practice of the aspects described below. The advantages described below will be realized and attained by elements and combinations particularly pointed out in the appended claims. It is to be understood that both the foregoing general description and the following detailed description are exemplary and explanatory only and are not restrictive.

### BRIEF DESCRIPTION OF THE FIGURE

The accompanying FIGURE, which is incorporated in and constitutes a part of this specification, illustrates several aspects described below.

FIG. 1 is a group of coronal MRI time-course images following injection of Gd-sucrose scaffold into the mammary fat-pad (MFP) of mouse bearing ALN metastasis (top panels). The agent has cleared the ALN by 75 mins. Axial view showing agent contrast in ALN (bottom left) and ex vivo images showing ALN metastasis contrast in injected versus control tissues (bottom right).

### DETAILED DESCRIPTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, reference to “the agent” includes mixtures of two or more such agents, and the like.

“Optional” or “optionally” means that the subsequently described event or circumstance can or cannot occur, and that the description includes instances where the event or circumstance occurs and instances where it does not.

By “reduce” or other forms of the word, such as “reducing” or “reduction,” is meant lowering of an event or characteristic (e.g., tumor growth, metastasis). It is understood that this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, delaying spread (e.g., metastasis) of the cancer, delaying occurrence or recurrence of cancer, delay or slowing of cancer progression, amelioration of the cancer state, and remission (whether partial or total).

The term “patient” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

### Compounds

Carbonic anhydrase IX and XII (CAIX and CAXII) are identified as targets that are expressed in nearly 100% of breast cancer lymph node metastases, but are not expressed in normal/unaffected lymph nodes or surrounding tissues (Tafreshi N K, et al., “Noninvasive detection of breast cancer lymph node metastasis using carbonic anhydrases IX and XII targeted imaging probes.” Clin. Cancer Res. 2012; 18(1):207-19). In a proof of principle study where near-infrared fluorescent (NIRF) dye was conjugated to monoclonal antibodies specific for these markers and the conjugates were tested in animal models of breast cancer ALN metastasis, these imaging conjugates are shown to transit the mammary fat pad and are retained in lymph node metastases that express the target protein (Id.). As few as 1000 cells are detectable in vivo. A scaffold bearing eight terminal alkyne groups was synthesized from sucrose and copies of an azide-terminated Gd-DOTA complex were attached via copper(I)-catalyzed azide-alkyne cycloaddition (Martinez G V, et al., “Demonstration of a sucrose-derived contrast agent for magnetic resonance imaging of the GI tract.” Bioorg. Med. Chem. Lett. 2013; 23(7):2061-4). This scaffold was injected into the mammary fat pad of mice and, by using MRI, demonstrated its clearance through the lymphatics, providing contrast to the ALNs. The scaffold cleared from the nodes within 2 hours post administration. Moreover, pyrazoles have been identified with high affinity and selectivity for both CAIX and CAXII (Kumar S, et al., “Pyrazolylbenzo[d]imidazoles as new potent and selective inhibitors of carbonic anhydrase isoforms hCA IX and XII.” Bioorg. Med. Chem. 2016; 24(13):2907-13), and ⁶⁸Ga-CA-IX inhibitor conjugates have been used by the same group for in vivo PET (positron-emission tomography) imaging.

Radio- and fluorescent-labeling of a pyrazole with CAIX and CAXII specificity can be used for both gamma-detection and fluorescence imaging of ALN metastasis with high sensitivity (e.g., no false negatives) and high specificity (e.g., low false positives), sparing the majority of patients from a nonselective and morbid complete ALN dissection; or in the case of pathologically node negative disease, eliminating ALN biopsy altogether. Targeted identification of ALN metastasis can revolutionize the entire practice of breast cancer surgery, reducing the considerable morbidity of ALN dissection and eliminating the need for sentinel node excision altogether.

In specific aspects, disclosed herein are compounds having Formula I.

A compound having Formula I

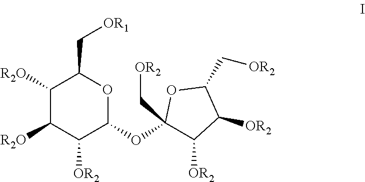

wherein
R₁ and R₂ are each independently chosen from (CH₂)ₙC≡CH and

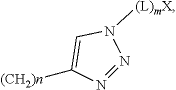

L is a linking moiety;
n is 1-8;
m is 0-8;
each X is independently chosen from R₃, OR₃, NH₂, NHR₃, and

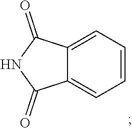 [SMILES: O=C1NC(=O)c2ccccc21]

and each R₃ is independently chosen from a detectable moiety or targeting moiety,
with the proviso that R₁ and R₂ are not both (CH₂)₄C≡CH. In specific examples, there is at least one R₃ that is a detectable moiety and at least one R₃ that is a targeting moiety.

In certain examples, the linker moiety can be from 1 to 12 atoms in length. For example, the linker moiety can be O, S, NH, R₄, OR₄, R₄O, OR₄O, C(O)R₄, R₄C(O), C(O)R₄C(O), C(O)OR₄, R₄OC(O), C(O)OR₄OC(O), C(O)R₄N, NR₄C(O), C(O)OR₄NH, NHR₄C(O)O, NHR₄, R₄NH, NHR₄NH, or C(O)NHR₄NHC(O), wherein R₄ is C₁-C₁₂ alkyl which can be optionally substituted with one or more substituents including halogen, alkoxyl, alkyl, alkenyl, cycloalkyl, cycloalkenyl, heterocycloalkyl, aryl, heteroaryl, amine, cyano, nitro, hydroxyl, carbonyl (C═O), acyl, carboxylic acid (—COOH), or amide (—CONH₂). In specific examples, the linker moiety can be methyl, ethyl, propyl, butyl, pentyl, hexyl, heptyl, octyl, nonyl, or decyl. In other examples, the linker moiety can be polyethylene oxide (CH₂CH₂O)ₘ, where m is from 1 to 8.

In further examples, n is 3, 4, or 5. In further examples, n is 4. In further examples, m is 4, 5, 6, 7, or 8. In further examples, m is 6. In other examples, m is 0.

In further examples, R₁ is the same as R₂. In other examples R₁ and R₂ are different. In further examples, R₁ is

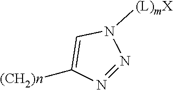

and R₂ is (CH₂)ₙC≡CH. In further examples, both R₁ and R₂ are

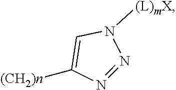

where each X is independently chosen from R₃, OR₃, NH₂, NHR₃, and

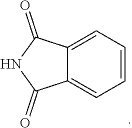 [SMILES: O=C1NC(=O)c2ccccc21]

In further examples, R₁ and R₂ are both

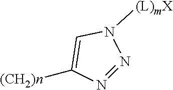

with each X═NH₂ or NHR₃.

In further examples, R₁ and R₂ are each independently selected from (CH₂)₄C≡CH,

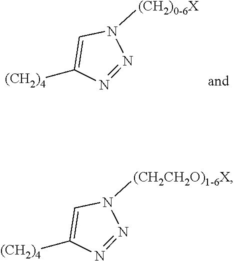

with the proviso that R₁ and R₂ are not both (CH₂)₄C≡CH.

In further example, R₁ is

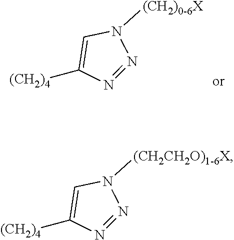

where X is R₃, OR₃, or NHR₃, where R₃ is a detectable moiety such as IRDye 800CW or a DOTA or DOTA-based chelated radionuclide.

In further example, R₁ is

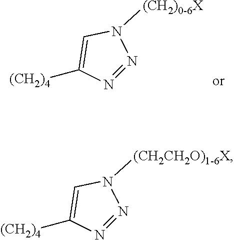

where X is NH₂, NHR³, or

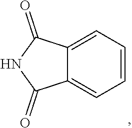

where R₃ is a detectable moiety such as IRDye 800CW or a DOTA or DOTA-based chelated radionuclide.

In further example, R₂ is

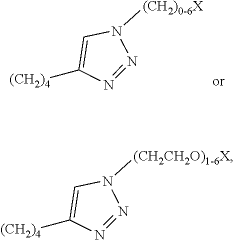

where m is 0 or 6, and X is R₃ or OR₃, where R₃ is a targeting moiety such as

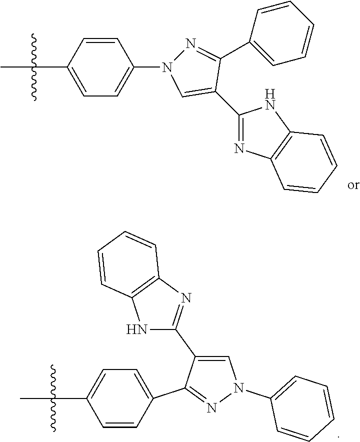 [SMILES: *c1ccc(-n2cc(-c3nc4ccccc4[nH]3)c(-c3ccccc3)n2)cc1 ; *c1ccc(-c2nn(-c3ccccc3)cc2-c2nc3ccccc3[nH]2)cc1]

In further examples, R₁ is

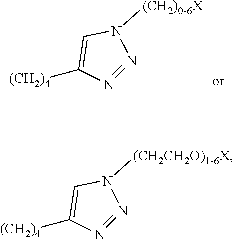

where X is R₃, OR₃, or NHR₃, where R₃ is a detectable moiety; and

### R₂ is

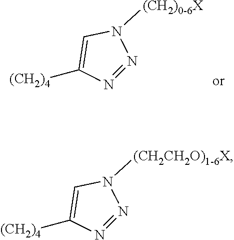

where X is R₃, OR₃, or NHR₃, where R₃ is a targeting moiety. The detectable moiety can be chosen from IRDye 800CW and DOTA or DOTA-based chelated radionuclide. In some examples, the targeting moiety is

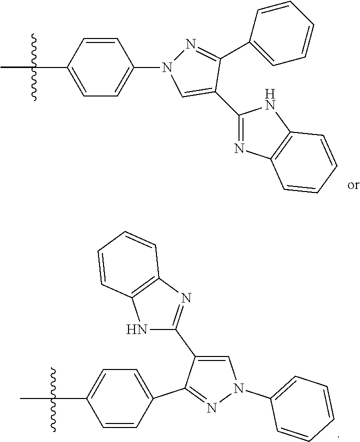 [SMILES: *c1ccc(-n2cc(-c3nc4ccccc4[nH]3)c(-c3ccccc3)n2)cc1 ; *c1ccc(-c2nn(-c3ccccc3)cc2-c2nc3ccccc3[nH]2)cc1]

Further examples of suitable detectable moieties include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, a isotope detectable spin resonance label, a paramagnetic moiety, a chromophore, or any combination thereof.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene, perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetrapheny metrylbenzoporphyrin; Pd (II) octaethylporphyrin ketone; Pt (II) octaethylporphyrin ketone; Pd (II) meso-tetra(pentafluorophenyl)porphyrin; Pt (II) meso-tetra (pentafluorophenyl) porphyrin; Ru (II) tris(4,7-diphenyl-1,10-phenanthroline) (Ru (dpp)₃); Ru (II) tris(1,10-phenanthroline) (Ru(phen)₃), tris(2,2′-bipyridine)ruthenium (II) chloride hexahydrate (Ru(bpy)₃); erythrosine B; fluorescein; eosin; iridium (III) ((N-methyl-benzimidazol-2-yl)-7-(diethylamino)-coumarin)); indium (III) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitrophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluorescein; eosin; 2′,7′-dichlorofluorescein; 5(6)-carboxy-fluorescein; carboxynaphtofluorescein; 8-hydroxypyrene-1,3,6-trisulfonic acid; semi-naphthorhodafluor; semi-naphthofluorescein; tris (4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; and dioctadecylcycloxacarbocyanine; derivatives or combinations thereof.

The detectable moiety can contain a radiolabel, also referred to herein as radioisotope. The radiolabel can also be a therapeutic moiety, i.e., a radiolabel comprising a therapeutic radionuclide such as, ⁹⁰Y, ¹⁷⁷Lu, ²¹³Bi, or ²²⁵Ac. Other examples of suitable radiolabels include, but are not limited to, metal ¹⁸F, ⁶⁴Cu, ⁶⁷Cu, ⁸⁹Zr, ¹¹¹In, ¹²⁴I, ¹²³I, ⁶⁷Ga, and ^{99m}Tc. In some embodiments, the radiolabel can be chelated by a macrocyclic molecule. Examples of such macrocyclic molecules include, but are not limited to, 2,2′,2″-(10-(2-((2,5-dioxopyrrolidin-1-yl)oxy)-2-oxoethyl)-1,4,7,10-tetraazacyclododecane-1,4,7-triyl)triacetic acid (DOTA)-based chelators, such as such as DTPA (diethylene triamine pentaacetic acid), DOTP (1,4,7,10-Tetraazacyclododecane-1,4,7,10-tetra(methylene phosphonic) acid), DOTMA, (1R, 4R, 7R, 10R)-α′α″α′″-Tetramethyl-1,4,7,10-tetraazacyclododecane-1,4,7,10-tetraacetic acid) tetrasodium salt, TETA, (1,4,8,11-Tetraazacyclotetradecane-1,4,8, 11-tetraacetic acid), DOTAM (1,4,7,10-Tetrakis(carbamoylmethyl)-1,4,7,10-tetraazacyclododecane), CB-TE2A (1,4,8,11-tetraazabicyclo[6.6.2]hexadecane-4, 11-dicetic acid), and NOTA ((1,4,7-triazacyclononane-N,N′,N″-triacetic acid), and derivatives or a combination thereof. In a specific example, the detectable moiety is a chelated ¹¹¹In radionuclide. In other examples, the detectable moiety is a chelated Gd.

The detectable moiety can contain a magnetic spin resonance label. Examples of suitable spin resonance label include free radicals such as nitroxide-stable free radicals. Stable free radicals of nitroxides are known in the art, see for example Keana, “Newer Aspects of Synthesis and Chemistry of Nitroxide Spin Labels”, Chemical Reviews, 1978, Vol. 78 No. 1, pp. 37-64, which disclosure is incorporated herein by reference. Suitable nitroxides include, but are not limited to, those derived from 2,2,6,6-tetramethylpiperidine-N-oxyl (TEMPO), 2,2,5,5-tetramethylpyrroline-N-oxyl, and 4,4-dimethyloxazolidine-N-oxyl which is a doxyl nitroxide. All of these compounds are paramagnetic and hence capable of excitation or changes in magnetic resonance energy levels and therefore provide imaging. Other nitroxides include, but are not limited to, doxyl nitroxides, proxyl nitroxides, azethoxyl nitroxides, imidazoline derived nitroxides, tetrahydrooxazine derived nitroxides, and the recently synthesized steroid nitroxides, and the like.

Spin labeling, as used herein, is understood to mean “spin label” as that is defined in the Keana article, namely when a nitroxide bearing molecule that is covalently attached to another molecule of interest, the nitroxide grouping does not significantly disturb the behavior of the system under study. Thus, the nitroxide molecule being paramagnetic, simply enhances the energy or excitation level subjected to the magnetic field during the magnetic resonance.

The detectable moiety can also be a MRI contrast agent such as a chelated Gd. Barium contrast agents can also be used. Further examples, include lanthanide contrast agents such as chelated Europium can be used.

The targeting moiety can be any agent that targets carbonic anhydrase IX and XII. Examples include pyrazoles, sulfonamides, peptides, monoclonal antibodies and antibody fragments that bind carbonic anhydrase IX and XII.

The disclosed compositions and methods can have the ability to reliably detect and selectively remove metastasis containing lymph nodes during surgery with low or no false-negatives. The disclosed compositions and methods combine emerging technologies, such as molecular targeting and NIRF fluorescence imaging for surgical guidance with the current standard of care, gamma wand detection, to allow real-time surgical detection of metastatic ALN for personalized patient care.

### Method of Making

Referring to Scheme 1 and starting from the sucrose-derived octaalkyne 1 (Martinez G V, et al., “Demonstration of a sucrose-derived contrast agent for magnetic resonance imaging of the GI tract.” Bioorg. Med. Chem. Lett. 2013; 23(7):2061-4), CuAAc reaction (Hein J E, et al., “Copper-catalyzed azide-alkyne cycloaddition (CuAAC) and beyond: new reactivity of copper(I) acetylides.” Chem. Soc. Rev. 2010; 39(4):1302-15) with an excess of 1-azido-6-phthalimidohexane, followed by hydrolysis of the phthalimide moieties, will produce octaamine 2. Reaction of 2 with an excess of commercially available IRDye 800CW NHS ester 3 will give the untargeted, fluorescently tagged compound 4. Alternatively, reaction of a large excess of 1 with one equivalent of 1-azido-6-phthalimidohexane will produce heptaalkyne 5 as a mixture of regioisomers (only one is shown). CuAAc reaction of 5 with an excess of pyrazole 6 (Kumar S, et al., “Pyrazolylbenzo[d]imidazoles as new potent and selective inhibitors of carbonic anhydrase isoforms hCA IX and XII.” Bioorg. Med. Chem. 2016; 24(13):2907-13) (inhibitor with hCA IX and XII selectivity), followed by phthalimide hydrolysis, will give amine 7. Reaction of 7 with NHS ester 3 will produce the targeted, fluorescently tagged compound 8. Alternatively, amine 7 can be coupled with DOTA, giving 9 which can be loaded with ¹¹¹In radionuclide. For radiolabeling, the precursor is dissolved 0.1M NaOAc buffer followed by addition of ¹¹¹InCl₃ solution to provide a final pH=5.5-6. The mixture is heated in a water bath at 60° C. for 30 min. Radiolabeling is monitored by HPLC. At completion, the reaction mixture is diluted with 1 mL of water then loaded onto a preparative HPLC column for purification (Scheme 2).

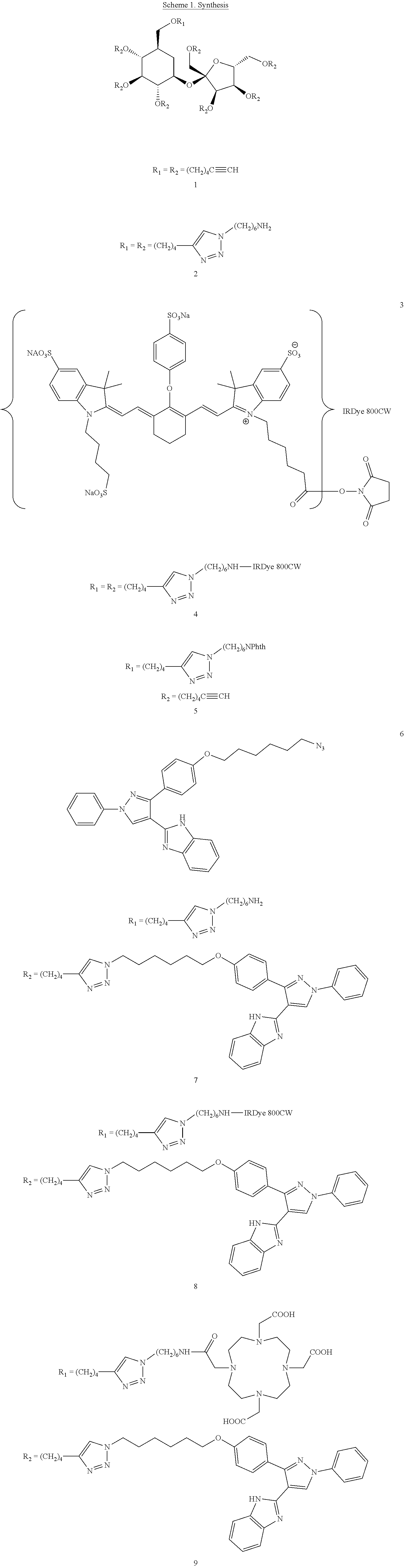

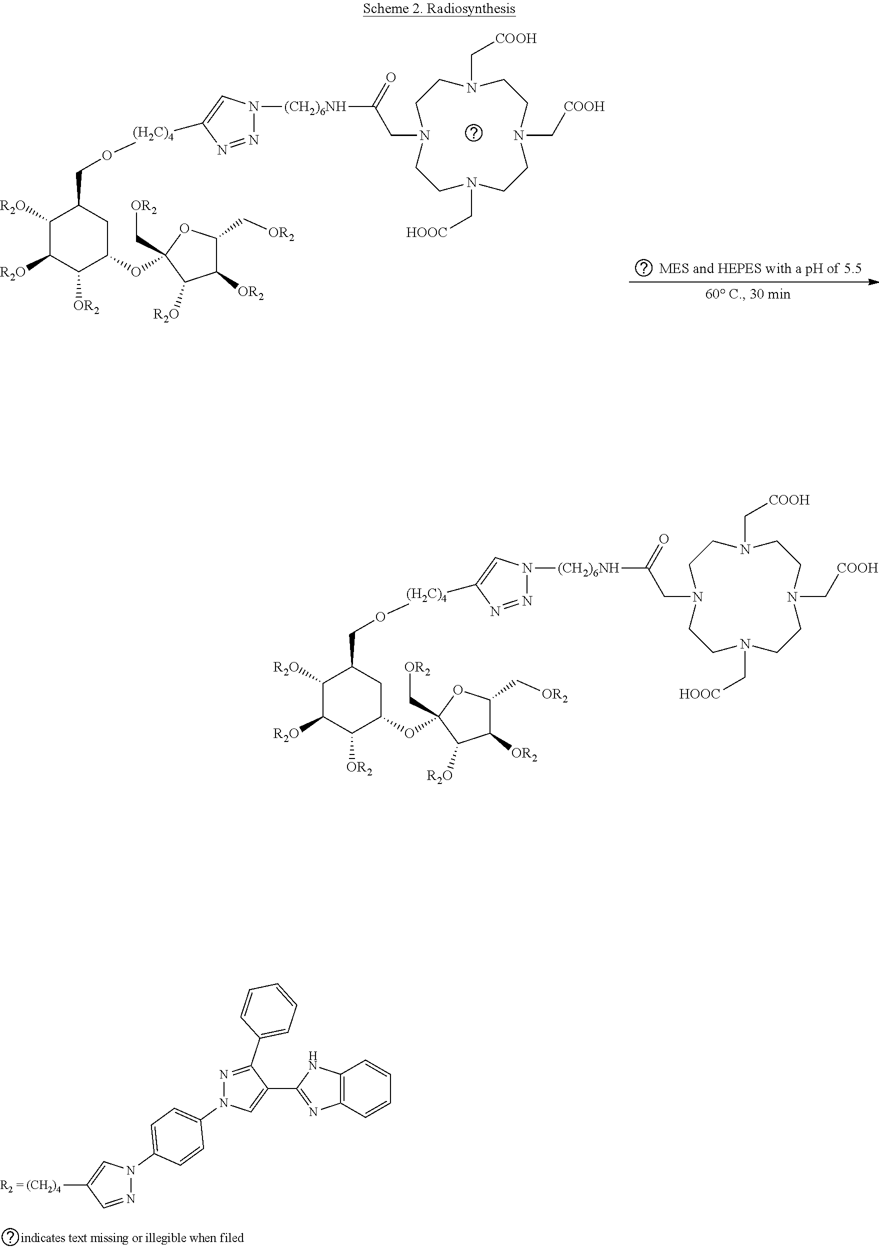

### Method of Use

Further provided herein are methods of imaging a cancer in a patient, comprising administering to the patient an effective amount of a compound or composition as disclosed herein, and imaging or detecting the compound or composition, or the detectable moiety thereon. The method can further comprise surgical procedures before or after administering the composition or compound disclosed herein. The methods can further comprise administering a second compound or composition, such as, for example, anticancer agents or anti-inflammatory agents. Additionally, the method can further comprise administering an effective amount of ionizing radiation to the subject. Also disclosed are methods of performing selective nodal excision that comprise administering to a patient a compound or composition disclosed herein and selectively excising a node. The methods can further comprise the imaging of the compound or composition or the detectable moiety thereon. Also disclosed are methods of detecting metastatic breast cancer lymph nodes that comprise administering to a patient a compound or composition disclosed herein and detecting the composition or composition or the detectable moiety thereon.

In certain examples the detectable moiety is a 1,4,7,10-tetraazacyclo-dodecane-1,4,7,10-tetraacetic acid (DOTA) or DOTA-based chelated radionuclide and detecting the detectable moiety is by gama wand, single photon emission computed tomography (SPECT) or positron emission tomography (PET) imaging. In other examples, the detectable moiety is a fluorescent moiety and detecting the detectable moiety is by fluorescence imaging.

The patient can be a human or other mammal, such as a primate (monkey, chimpanzee, ape, etc.), dog, cat, cow, pig, or horse, or other animals having cancer. The compounds disclosed herein are particularly suited for patients with breast cancer. However, other oncological disorders that are characterized by expression of carbonic anhydrase IX and XII can be treated. Specific examples of such oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Other examples of cancers that can be imaged according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

In specific examples, the cancer is breast cancer because CA-IX and CA-XII are cell-surface markers for breast cancer ALN metastasis. By mRNA expression profiling of breast cancer lymph node metastases and confirming protein expression via IHC staining of ALN metastases and surrounding normal patient tissues, it has been determined that either one or both of CA-IX and CA-XII were expressed in 100% of the ALN metastasis samples screened but not in surrounding tissues.

Carbonic anhydrase (CA) inhibitors have been attached to NIRF dye and ⁶⁸Ga-DOTA chelators for fluorescence and positron emission tomography (PET) molecular imaging. Thus the compounds disclosed herein can be used in fluorescence and PET imaging.

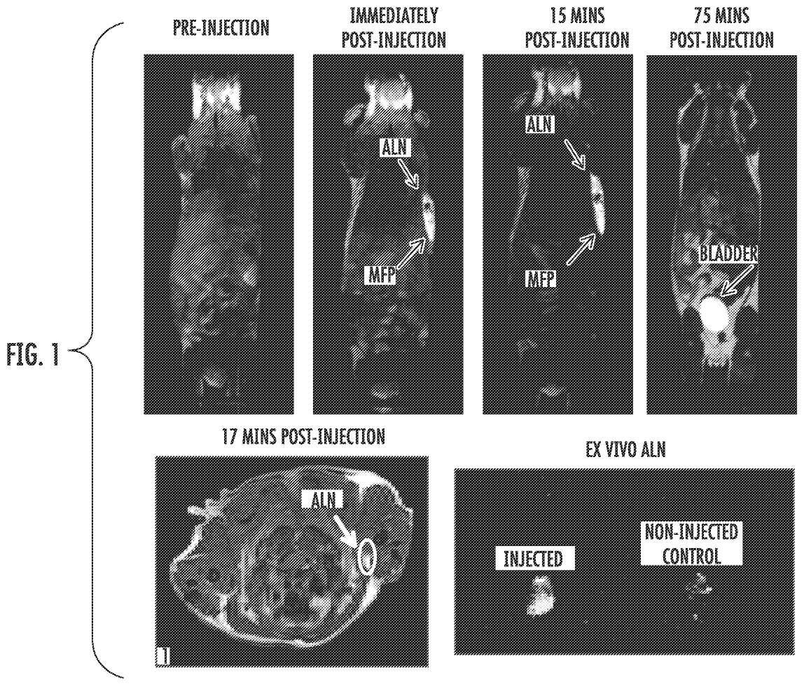

A problem with using antibody agents for targeting ALN has been the long clearance times. Such agents take days to fully clear the lymphatics via the mammary fat. Conversely, small molecule agents, e.g., ICG dye, have been problematic as they leak out of the lymphatic vessels and are taken directly into the blood stream. Hence, the standard of care uses colloids for ALN detection, where the colloids are large enough to stay in the lymphatic vessels but small enough to rapidly transit the vessels to the node. For development of a targeted imaging agent for ALN metastasis detection, a scaffold is needed that will 1) rapidly clear from the mammary fat via the lymphatics, 2) can be conjugated to a small molecule inhibitor that selectively binds metastasis targets for retention at sites of nodal metastasis, 3) can be conjugated to both radionuclide chelators and NIRF dyes for detection by gamma wand and real-time intraoperative fluorescence imaging, and 4) will rapidly clear from the lymph nodes in the absence of metastases. A sucrose-based scaffold that can carry multiple attachments, i.e. CA-IX and CA-XII selective inhibitors for targeting, and NIRF dyes and chelators for attachment of gamma-emitting radionuclide was developed herein (Martinez G V, et al., “Demonstration of a sucrose-derived contrast agent for magnetic resonance imaging of the GI tract.” Bioorg. Med. Chem. Lett. 2013; 23(7):2061-4). By magnetic resonance imaging in mouse ALN metastasis model, it has been demonstrated that this scaffold, when conjugated to a Gd-DOTA chelate and injected into the mammary fat, rapidly transits through the lymphatics, clearing the ALN within hours (FIG. 1). Also demonstrated herein is that when the agent is taken into the blood, it clears rapidly via the renal system.

For detection by gamma wand, ¹¹¹In or ⁶⁷Ga radionuclides can be used. ¹¹¹In or ⁶⁷Ga have gamma emissions similar to Tc-99m as all three can be used for detection by single-photon emission tomography (SPECT, PEM, MBI). Both ¹¹¹In and ⁶⁷Ga have long half-lives (67 h and 78.26 h, respectively) that are compatible with the timing of the disclosed procedure and allow for centralized production of the disclosed compounds and have proven chelation to DOTA carbonic anhydrase IX and XII.

### Administration

The disclosed compounds can be administered either sequentially or simultaneously in separate or combined pharmaceutical formulations. When one or more of the disclosed compounds is used in combination with a second therapeutic agent, the dose of each compound can be either the same as or differ from that when the compound is used alone. Appropriate doses will be readily appreciated by those skilled in the art.

The term “administration” and variants thereof (e.g., “administering” a compound) in reference to a compound as described herein means introducing the compound or a prodrug of the compound into the system of the animal in need of treatment. When a compound as described herein or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.10% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10(18):17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation; East Hanover, N.J.) and HERCEPTIN (Genentech, Inc.; South San Francisco, Calif.), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², Bi²¹³, Ac²²⁵ etc.), and toxins of bacterial, fungal, plant, or animal origin (e.g., ricin, botulinum toxin, anthrax toxin, aflatoxin, jellyfish venoms (e.g., box jellyfish, etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

### EXAMPLES

### Example 1. Development of a Radiolabeled Conjugate for Detection with Gamma Wand

A conjugate can be developed that is rapidly cleared from tumor proximal mammary fat via the lymphatics, is targeted to specifically bind CAIX and CAXII, and is radiolabeled for detection by gamma wand. Precursor compound can be by conjugation of a CAIX and CAXII specific pyrazole and a DOTA chelate to a sucrose scaffold. The precursor can be labeled with non-radioactive indium (In) and can be analyzed by HPLC to determine purity. Binding avidity can be determined by an established competition assay. Radiolabeling of precursors can generate [¹¹¹In]In-DOTA-pyrazole-scaffold conjugates (¹¹¹In, T_{1/2}, 67.32 h). Alternatively, ⁶⁷Ga can be used and chelated to the scaffold conjugated. ⁶⁷Ga is also chelated by DOTA, has a 78.26 h half-life and is readily available. Radiochemical yield, purity and biostability in human plasma at 37° C. can be determined using radio-TLC and radio-HPLC. In vitro cellular uptake can be determined using human breast tumor cells with endogenous CAIX and CAXII expression (MCF-7, MDA-mb-231 and ZR-75.1). Existing orthotopic mouse models of breast cancer lymph node metastasis with endogenous CA expression can be used for in vivo studies to determine selectivity for ALN metastases. During a post-administration time-course, the clinical Neoprobe 2300 wand can be used for in vivo gamma counting; and pre-clinical micro-SPECT/CT images can be acquired to determine agent accumulation in metastases relative to surrounding tissues. Blocking studies can be used to determine agent-specificity. Collected tissues and metastases can be fixed, allowed to decay, sectioned and IHC stained for CAIX and CAXII.

### Example 2. Development of a NIRF Dye Conjugate

As described above for the nuclear medicine agent, a CAIX/CAXII targeted infrared imaging agent can be developed based on pyrazole inhibitors and a sucrose scaffold, except that instead of metal chelation, the IRDye800CW dye (Licor) can be conjugated for use in real-time fluorescence imaging detection of probe uptake. As above, in vitro cellular uptake and ALN metastasis imaging studies can be performed using the same animal models. Except that the measurements can be performed using fluorescence imaging instrumentation, the In Vivo Xtreme (Bruker) system for small animal multi-spectral unmixing reflectance imaging and the SurgVision Clinical Real-Time Fluorescence imaging platform.

1. A compound having Formula I: 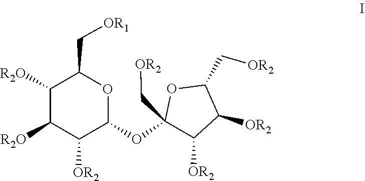
wherein
R₁ and R₂ are each independently chosen from (CH₂)ₙC≡CH and 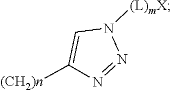
L is a linking moiety;
n is 1-8;
m is 0-8;
each X is independently chosen from R₃, OR₃, NH₂, NHR₃, and 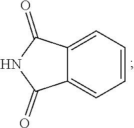 [SMILES: O=C1NC(=O)c2ccccc21]
and
each R₃ is independently chosen from a detectable moiety or targeting moiety,
with the proviso that R₁ and R₂ are not both (CH₂)₄C≡CH.

2. The compound of claim 1, wherein there is at least one R₃ that is a detectable moiety and at least one R₃ that is a targeting moiety.

3. The compound of claim 1, wherein n is 3, 4, or 5.

4. The compound of claim 1, wherein n is 4.

5. The compound of claim 1, wherein m is 4, 5, 6, 7, or 8.

6. The compound of claim 1, wherein m is 6.

7. The compound of claim 1, wherein m is 0.

8. The compound of claim 1, wherein n is 4 and m is 0 or 6.

9. The compound of claim 1, wherein L is from 1 to 12 atoms in length.

10. The compound of claim 1, wherein L is O, S, NH, R₄, OR₄, R₄O, OR₄O, C(O)R₄, R₄C(O), C(O)R₄C(O), C(O)OR₄, R₄OC(O), C(O)OR₄OC(O), C(O)R₄N, NR₄C(O), C(O)OR₄NH, NHR₄C(O)O, NHR₄, R₄NH, NHR₄NH, or C(O)NHR₄NHC(O), wherein R₄ is C₁-C₁₂ alkyl which can be optionally substituted with one or more substituents including halogen, alkoxyl, alkyl, alkenyl, cycloalkyl, cycloalkenyl, heterocycloalkyl, aryl, heteroaryl, amine, cyano, nitro, hydroxyl, carbonyl (C═O), acyl, carboxylic acid (—COOH), or amide (—CONH₂).

11. The compound of claim 1, wherein L is methyl, ethyl, propyl, butyl, pentyl, hexyl, heptyl, octyl, nonyl, or decyl.

12. The compound of claim 1, wherein L is (CH₂CH₂O)ₘ, where m is from 1 to 8.

13. The compound of claim 1, wherein R₁ and R₂ are different.

14. The compound of claim 1, wherein
R₁ is 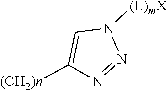
and R₂ is (CH₂)ₙC≡CH.

15. The compound of claim 1, wherein both R₁ and R₂ are 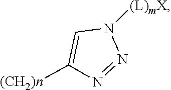
where each X is independently chosen from R₃, OR₃, NH₂, NHR₃, and

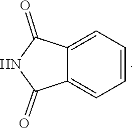 [SMILES: O=C1NC(=O)c2ccccc21]

16. The compound of claim 1, wherein both R₁ and R₂ are 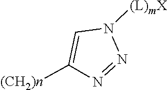
with each X═NH₂ or NHR₃.

17. The compound of claim 1, wherein R₁ and R₂ are each independently selected from (CH₂)₄C≡CH,

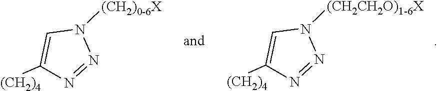

18. The compound of claim 1, wherein R₁ is 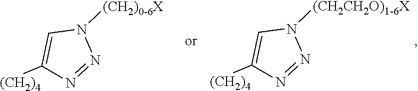
where X is NH₂, NHR₃, or 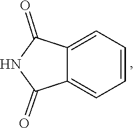
where R₃ is IRDye 800CW or 1,4,7,10-tetraazacyclo-dodecane-1,4,7,10-tetraacetic acid (DOTA) or DOTA-based chelated radionuclide.

19. The compound of claim 1, wherein R₂ is 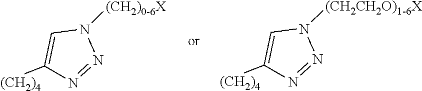
where m is 0 or 6, and X is R₃ or OR₃, where R₃ is

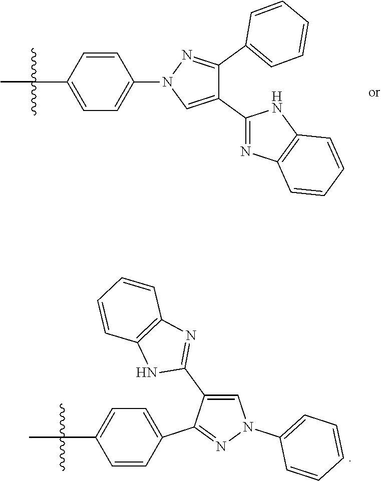 [SMILES: *c1ccc(-n2cc(-c3nc4ccccc4[nH]3)c(-c3ccccc3)n2)cc1 ; *c1ccc(-c2nn(-c3ccccc3)cc2-c2nc3ccccc3[nH]2)cc1]

20. The compound of claim 1, wherein R₁ is 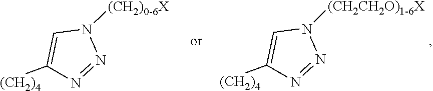
where X is R₃, OR₃, or NHR₃, where R₃ is a detectable moiety; and
R₂ is 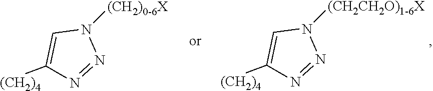
where X is R₃, OR₃, or NHR₃, where R₃ is a targeting moiety.

21. The compound of claim 1, wherein the detectable moiety is chosen from IRDye 800CW and 1,4,7,10-tetraazacyclo-dodecane-1,4,7,10-tetraacetic acid (DOTA) or DOTA-based chelated radionuclide.

22. The compound of claim 1, wherein the detectable moiety is a chelated radionuclide, wherein the radionuclide is selected from ⁹⁰Y, ¹⁷⁷Lu, ¹⁸F, ⁶⁴Cu, ⁶⁷Cu, ⁸⁹Zr, ¹¹¹In, ¹²⁴I, ¹²³I, Bi²¹³, Ac²²⁵, ⁶⁷Ga, or ^{99m}Tc.

23. The compound of claim 1, wherein the targeting moiety is

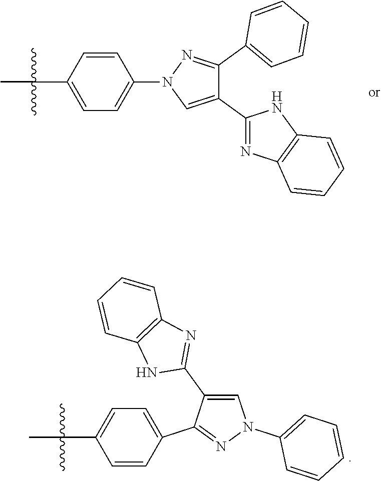 [SMILES: *c1ccc(-n2cc(-c3nc4ccccc4[nH]3)c(-c3ccccc3)n2)cc1 ; *c1ccc(-c2nn(-c3ccccc3)cc2-c2nc3ccccc3[nH]2)cc1]

24. The compound of claim 1, wherein the compound has two or more different detectable moieties.

25. The compound of claim 24, wherein the detectable moieties are a fluorescent moiety and a chelated radionuclide.

26. The compound of claim 1, wherein the compound has a detectable moiety and a targeting moiety.

27. A method of performing selective nodal excision, comprising:
administering to a patient a compound of claim 1, and selectively excising a node.

28. A method of imaging a cancer in a patient, comprising:
administering to the patient an effective amount of a compound of claim 1 and detecting the detectable moiety.

29. A method of detecting metastatic breast cancer lymph nodes, comprising: administering to a patient a compound of claim 1 and detecting the detectable moiety.

30. The method of claim 29, wherein the patient has breast cancer.

31. The method of claim 29, wherein the detectable moiety is a 1,4,7,10-tetraazacyclo-dodecane-1,4,7,10-tetraacetic acid (DOTA) or DOTA-based chelated radionuclide and detecting the detectable moiety is by single photon emission computed tomography (SPECT) or positron emission tomography (PET) imaging.

32. The method of claim 29, wherein the detectable moiety is a fluorescent moiety and detecting the detectable moiety is by fluorescence imaging.
